(19) United States
(12) Patent Application Publication
(10) Pub. No.: US 2023/0148321 A1
(43) Pub. Date: May 11, 2023

(54) METHOD FOR ARTIFICIAL INTELLIGENCE (AI) MODEL SELECTION

(71) Applicant: PRESAGEN PTY LTD, Adelaide (AU)

(72) Inventors: Jonathan Michael MacGillivray HALL, Adelaide (AU); Donato PERUGINI, Adelaide (AU); Michelle PERUGINI, Adelaide (AU); Tuc Van NGUYEN, Adelaide (AU); Milad Abou DAKKA, Adelaide (AU)

(21) Appl. No.: 17/916,288

(22) PCT Filed: Mar. 30, 2021
(86) PCT No.: PCT/AU2021/000029

§ 371 Date: Sep. 30, 2022

(30) Foreign Application Priority Data
Apr. 3, 2020 (AU) 202090142

(51) Int. Cl. G06N 20/20

(52) U.S. Cl. CPC G06N 20/20; G16H 50/50

(57) ABSTRACT

Computational methods and systems for training Artificial Intelligence (AI) models with improved translatability or generalisability (robustness) comprises training a plurality of Artificial Intelligence (AI) models using a common validation dataset over a plurality of epochs. During training of each model, at least one confidence metric is calculated at one or more epochs, and, for each model, the best confidence metric value over the plurality of epochs, and the associated epoch number at the best confidence metric is stored. An AI model is then generated by selecting at least one of the plurality of trained AI models based on the stored best confidence metric and calculating a confidence metric for the selected at least one trained AI model applied to a blind test set. The resultant AI model is saved and deployed if the best confidence metric exceeds an acceptance threshold.

### PRIORITY DOCUMENTS

The present application claims priority from Australian Provisional Patent Application No. 2020901042 titled “METHOD FOR ARTIFICIAL INTELLIGENCE (AI) MODEL SELECTION” and filed on 3 Apr. 2020, the content of which is hereby incorporated by reference in its entirety.

### TECHNICAL FIELD

The present disclosure relates to Artificial Intelligence. In a particular form the present disclosure relates to methods for training AI models and classifying data.

### BACKGROUND

Advancements in artificial intelligence (AI) has enabled the development of new products that are restructuring businesses and reshaping the future of many critical industries, including healthcare. Underlying these changes has been the rapid growth of machine learning and deep learning (DL) technologies.

Both machine learning and deep learning are two subsets of Artificial intelligence (AI). Machine learning is a technique or algorithm that enables machines to self-learn a task (e.g., create predictive models), without human intervention or being explicitly programmed. Supervised machine learning (or supervised learning) is a classification technique that learns patterns in labelled (training) data, where the labels or annotations for each datapoint relates to a set of classes, in order to create (predictive) AI models that can be used to classify new unseen data. In the context of this specification, AI will be used to refer to both machine learning and deep learning methods.

Using identification of embryo viability in IVF as an example, images of an embryo can be labelled “viable” if the embryo led to a pregnancy (viable class) and non-viable if the embryo did not lead to a pregnancy (non-viable class). Supervised learning can be used to train on a large dataset of labelled embryo images in order to learn patterns that are associated with viable and non-viable embryos. These patterns are incorporated in an AI model. The AI model can then be used to classify new unseen images to identify if an embryo (via inferencing on the embryo image) is likely to be viable (i.e., is likely to lead to a pregnancy and thus is a candidate for being transferred to the patient in the IVF treatment) or non-viable (i.e., will not likely lead to a pregnancy and thus should not be transferred to the patient).

While deep learning is similar to machine learning in terms of learning objective, it goes beyond statistical machine learning models to better imitate the function of a human neural system. Deep learning models typically consist of artificial “neural networks” that contain numerous intermediate layers between input and output, where each layer is considered a sub-model, each providing a different interpretation of the data. While the machine learning commonly only accepts structured data as its input, deep learning, on the other hand, does not necessarily need structured data as its input. For example, in order to recognize an image of a dog and a cat, a traditional machine learning model needs user-predefined features from those images. Such a machine learning model will learn from certain numeric features as inputs and can then be used to identify features or objects from other unknown images. The raw image is sent through the deep learning network, layer by layer, and each layer would learn to define specific (numeric) features of the input image.

To train an AI model (including machine learning model and/or deep learning models), the following steps are normally performed:

a) Exploring the data, in the context of the problem domain and desired AI solution or application, which may involve identifying what kind of problem is being solved, e.g., a classification problem or a segmentation problem, and then precisely defining the problem to be solved, e.g., exactly what subset of data is to be used for training the model, and what categories the model will output results into;

b) Pre-processing the data, which includes data quality techniques/data cleaning to remove any label noise or bad data and preparing the data so it is ready to be utilized for AI training and validation;

c) Extract features if required by model (e.g., by using Computer Vision methods);

d) Choosing the model configuration, including model type, model architecture and machine learning hyper-parameters;

e) Splitting the data into training dataset, validation dataset and/or test dataset;

f) Training the AI model by using machine learning and/or deep learning algorithms on the training dataset; typically, during the training process, many models are produced by adjusting and tuning the model configurations in order to optimize the performance of model according to an accuracy metric; each training iteration is referred to as an epoch, with the accuracy estimated and model updated at the end of each epoch;

g) Choosing the best “final” model, or an ensemble of models, based on the model's performance on the validation dataset; the model is then applied to the “unseen” test dataset to validate the performance of the final AI model.

The machine learning or deep learning algorithm finds the patterns in the training data and maps that to the target. The trained model that results from this process is then able to capture these patterns.

As AI-powered technologies have become more prevalent, the demand for quality (e.g., accurate) AI prediction models has become clearer. The state of the literature on machine learning applications for classification of images (the field of computer vision) is predominantly focused on accuracy, as measured by the total number of correctly identified images into their categories, divided by the total number of images, on a blind test set. While in the specific case of extremely large datasets (tens to hundreds of thousands of images), and very well-contained problem sets, accuracy is a useful metric for establishing the performance of a model, many commercial applications of machine learning are suffering from issues of lack of generalizability (i.e., robustness) needed for the AI to scale and apply to different and diverse users globally, or failure to translate onto data sourced from a real industry dataset.

One of the reasons for this discrepancy between performance on standard artificial and well-curated datasets and real industry performance is that models tend to be ‘brittle’ or fail to generalize (or translate) to a dataset that extends outside the constrained narrow region of applicability, from the set that the model was trained on. Features of datasets, such as handling of bad data, poorly-labelled or misleading data and adversarial examples, while studied in the literature, do not typically feature in key computer vision competitions (e.g., Kaggle), and or their industry-specific counter parts, and therefore many techniques are not typically implemented as part of a protocol for training, validating and testing a robust and scalable AI model (which is needed for a commercially scalable AI product), and which metrics are the most suitable.

This is especially true in specific industries such as healthcare/medical image datasets, which differ from other well-studied computer vision datasets in a number of ways. First, medical images can contain significant information in very fine detail associated with features in the image, and the distribution of this information can be different from standard image datasets. This means that while transfer learning is a useful technique that has shown much benefit to medical applications, it is not alone sufficient, and re-training on a new, medical training set (specific to the problem at hand) must be accomplished on a medical dataset to be able to demonstrate predictive power.

Second, high quality and well-labelled medical data is usually much more scarce than other kinds of image data, meaning that using a coarse, single metric such as accuracy will be potentially vulnerable to either a) large statistical uncertainty due to a small validation and test set available for reporting metrics, and/or b) strong dependency of the model performance on the details of the distribution of the model's outputs, i.e., scores for classifying the images. This scarcity of high-quality, well-labelled medical data means that greater care must be taken in understanding the distribution of the model's outputs, its prediction scores, and whether the distribution is good. It also requires care in understanding other key metrics that are demonstrated to be better indicators of translatability to a new blind (unseen) dataset, or a double-blind dataset (a blind dataset that has been sourced from a clinic, locality or dataset with a different source or distribution from the training and validation set).

The focus on accuracy as a single metric that defines performance of an AI model in the field, at the expense of all other metrics, can have adverse consequences, as such AI models or AI products often fail to generalize well to new datasets thus leading to poor decision-making outcomes when used in practice.

There is thus a need to provide methods for generating AI models that perform well on new datasets (i.e., generalize well), or at least providing a useful alternative to existing methods.

### SUMMARY

A computational method for generating an Artificial Intelligence (AI) model, the method comprising:

training a plurality of Artificial Intelligence (AI) models using a common validation dataset over a plurality of epochs, wherein during training of each model, at least one confidence metric is calculated at one or more epochs, and, for each model, the best confidence metric value over the plurality of epochs, and the associated epoch number at the best confidence metric is stored;

generating an AI model comprising:

selecting at least one of the plurality of trained AI models based on the stored best confidence metric;

calculating a confidence metric for the selected at least one trained AI model applied to a blind test set; and

deploying the AI model if the best confidence metric exceeds an acceptance threshold.

In a further form, at least one confidence metric is calculated at each epoch.

In one form generating an AI model comprises generating an ensemble AI model using at least two of the plurality of trained AI models based on the stored best confidence metrics, and the ensemble model uses a confidence based voting strategy.

In a further form, generating an ensemble AI model comprises:

selecting at least two of the plurality of trained AI models based on the stored best confidence metric;

generating a plurality of distinct candidate ensemble models wherein each candidate ensemble model combines the results of the selected at least two of the plurality of trained AI models according to a confidence based voting strategy;

calculating the confidence metric for each candidate ensemble model applied to a common ensemble validation dataset;

selecting a candidate ensemble model from the plurality of distinct candidate ensemble models and calculating a confidence metric for the selected candidate ensemble model applied to a blind test set;

In one form, the common ensemble validation dataset may be the common validation dataset, or the common ensemble validation dataset may be an intermediate test set not used in training the plurality of Artificial Intelligence (AI) models.

In one form, the confidence based voting strategy may be selected from the group consisting of maximum confidence, mean confidence, majority-mean confidence, majority-max confidence, median confidence, or weighted mean confidence.

In one form generating an AI model comprises generating a student AI model using a distillation method to train the student model using at least two of the plurality of trained AI models using at least one confidence metric.

In one form selecting at least one of the plurality of trained AI models based on the stored best confidence metric comprises: selecting at least two of the plurality of trained AI models, comparing each of at least two of the plurality of trained AI models using a confidence based metric, and selecting the best trained AI models based on the comparison.

In one form, the at least one confidence metric comprises one or more of Log loss, combined class Log loss, combined data-source Log loss, combined class and data-source Log loss.

In one form, a plurality of assessment metrics are calculated and selected from the group consisting of accuracy, Mean class accuracy, sensitivity, specificity, a confusion matrix, Sensitivity-to-specificity ratio, precision, negative predictive value, balanced accuracy, Log loss, combined class Log loss, combined data-source Log loss, combined class and data-source Log loss, tangent score, bounded tangent score, per-class ratio of tangent score vs Log Loss, Sigmoid score, epoch number, mean of square error (MSE), root MSE, mean of average error, mean average precision (mAP), confidence score, Area-Under-the-Curve (AUC) threshold, Receiver Operating Characteristic (ROC) curve threshold, Precision-Recall curve. In a further form, the plurality of assessment metrics comprises a primary metric and at least one secondary metric, wherein the primary metric is a confidence metric, and the at least one secondary metric are used as tiebreaker metrics.

In one form, the plurality of AI models comprise a plurality of distinct model configurations, wherein each model configuration comprises a model type, a model architecture, and one or more pre-processing methods. In a further form, the one or more pre-processing methods may comprise segmentation, and the plurality of AI models comprises at least one AI model applied to unsegmented images, and at least one AI model applied to segmented images. In another form, the one or more pre-processing methods may comprise one or more computer vision pre-processing methods.

Embodiments of the method may be used in healthcare applications and thus in one form, the validation dataset is a healthcare dataset comprising a plurality of healthcare images.

According to a second aspect, there is provided a computational system comprising one or more processors, one or more memories, and a communications interface, wherein the one or more memories store instructions for configuring the one or more processors to computationally generate an Artificial Intelligence (AI) model according to the method of the first aspect. The computational system may be a cloud based system. According to a third aspect, there is provided a computational system comprising one or more processors, one or more memories, and a communications interface, wherein the one or more memories are configured to store an AI model trained using the method of the first aspect, and the one or more processors are configured to receive input data via the communications interface, process the input data using the stored AI model to generate a model result, and the communications interface is configured to send the model result to a user interface or data storage device

### BRIEF DESCRIPTION OF DRAWINGS

Embodiments of the present disclosure will be discussed with reference to the accompanying drawings wherein:

FIG. 1A is a schematic flowchart of the generation of an Artificial Intelligence (AI) model according to an embodiment;

FIG. 1B is a schematic flowchart of the generation of an ensemble Artificial Intelligence (AI) model according to an embodiment;

FIG. 2A is schematic architecture diagram of cloud based computation system configured to generate and use an AI model according to an embodiment;

FIG. 2B is a schematic flowchart of a model training process on a training server according to an embodiment;

FIG. 3 show the Score and Score gradient for the metrics Accuracy, Log Loss, Tangent Score and Sigmoid Score with respect to C, which provides a measure of the marginal sensitivities of the various metrics;

FIG. 4A is a plot of a histogram associated with the distribution of scores using Recall as the primary metric of positive pregnancy (viable) embryos for a single machine learning model on a validation set, with correct model predictions in bars with thick forward diagonal lines—True Positives, and incorrect model predictions in bars with thin rearward diagonal lines—False Negatives;

FIG. 4B is a plot of a histogram associated with the distribution of scores using Recall as the primary metric of negative pregnancy (non-viable) embryos for a single machine learning model on a validation set, with correct model predictions in bars with thick forward diagonal lines—True Negatives, and incorrect model predictions in bars with thin rearward diagonal lines—False Positives;

FIG. 4C is a plot of a histogram associated with the distribution of scores using Recall as the primary metric of positive pregnancy (viable) embryos for a single machine learning model on a combined blind/double-blind test set, with correct model predictions in bars with thick forward diagonal lines—True Positives, and incorrect model predictions in bars with thin rearward diagonal lines—False Negatives;

FIG. 4D is a plot of a histogram associated with the distribution of scores using Recall as the primary metric of negative pregnancy (non-viable) embryos for a single machine learning model on a combined blind/double-blind test set, with correct model predictions in bars with thick forward diagonal lines—True Negatives, and incorrect model predictions in bars with thin rearward diagonal lines—False Positives;

FIG. 5A is a plot of a histogram associated with the distribution of scores of positive pregnancy (viable) embryos of an Ensemble model, chosen based on Balanced Accuracy, on a shared validation set, with correct model predictions in green—True Positives, and incorrect model predictions in bars with thin rearward diagonal lines—False Negatives;

FIG. 5B is a plot of a histogram associated with the distribution of scores of negative pregnancy (non-viable) embryos of an Ensemble model, chosen based on Balanced Accuracy, on a shared validation set, with correct model predictions in bars with thick forward diagonal lines—True Negatives, and incorrect model predictions in bars with thin rearward diagonal lines—False Positives;

FIG. 5C is a plot of a histogram associated with the distribution of scores of positive pregnancy (viable) embryos of an Ensemble model, chosen based on Balanced Accuracy, on a shared blind test set, with correct model predictions in bars with thick forward diagonal lines—True Positives, and incorrect model predictions in bars with thin rearward diagonal lines—False Negatives;

FIG. 5D is a plot of a histogram associated with the distribution of scores of negative pregnancy (non-viable) embryos of an Ensemble model, chosen based on Balanced Accuracy, on a shared blind test set, with correct model predictions in bars with thick forward diagonal lines—True Negatives, and incorrect model predictions in bars with thin rearward diagonal lines—False Positives;

FIG. 6A is a plot of a histogram associated with the distribution of scores of positive pregnancy (viable) embryos of an Ensemble model, chosen based on Log Loss, on a shared validation set, with correct model predictions in bars with thick forward diagonal lines—True Positives, and incorrect model predictions in bars with thin rearward diagonal lines—False Negatives;

FIG. 6B is a plot of a histogram associated with the distribution of scores of negative pregnancy (non-viable) embryos of an Ensemble model, chosen based on Log Loss, on a shared validation set, with correct model predictions in bars with thick forward diagonal lines—True Negatives, and incorrect model predictions in bars with thin rearward diagonal lines—False Positives;

FIG. 6C is a plot of a histogram associated with the distribution of scores of positive pregnancy (viable) embryos of an Ensemble model, chosen based on Log Loss, on a shared blind test set, with correct model predictions in bars with thick forward diagonal lines—True Positives, and incorrect model predictions in bars with thin rearward diagonal lines—False Negatives; and

FIG. 6D is a plot of a histogram associated with the distribution of scores of negative pregnancy (non-viable) embryos of an Ensemble model, chosen based on Log Loss, on a shared blind test set, with correct model predictions in bars with thick forward diagonal lines—True Negatives, and incorrect model predictions in bars with thin rearward diagonal lines—False Positives.

FIG. 7A is a plot of a histogram associated with the distribution of scores using Per-Class Ratio of Tangent Score vs Log Loss as the primary metric of positive pregnancy (viable) embryos for a single machine learning model on a validation set, with correct model predictions in bars with horizontal lines—True Positives, and incorrect model predictions in black filled bars—False Negatives;

FIG. 7B is a plot of a histogram associated with the distribution of scores using Per-Class Ratio of Tangent Score vs Log Loss as the primary metric of negative pregnancy (non-viable) embryos for a single machine learning model on a validation set, with correct model predictions in in bars with horizontal lines—True Negatives, and incorrect model predictions in black filled bars—False Negatives;

FIG. 7C is a plot of a histogram associated with the distribution of scores using Per-Class Ratio of Tangent Score vs Log Loss as the primary metric of positive pregnancy (viable) embryos for a single machine learning model on a combined blind/double-blind test set, with correct model predictions in bars with horizontal lines—True Positives, and incorrect model predictions in black filled bars—False Negatives; and

FIG. 7D is a plot of a histogram associated with the distribution of scores using Per-Class Ratio of Tangent Score vs Log Loss as the primary metric of negative pregnancy (non-viable) embryos for a single machine learning model on a combined blind/double-blind test set, with correct model predictions in bars with horizontal lines—True Negatives, and incorrect model predictions in black filled bars—False Negatives.

In the following description, like reference characters designate like or corresponding parts throughout the figures.

### DETAILED DESCRIPTION

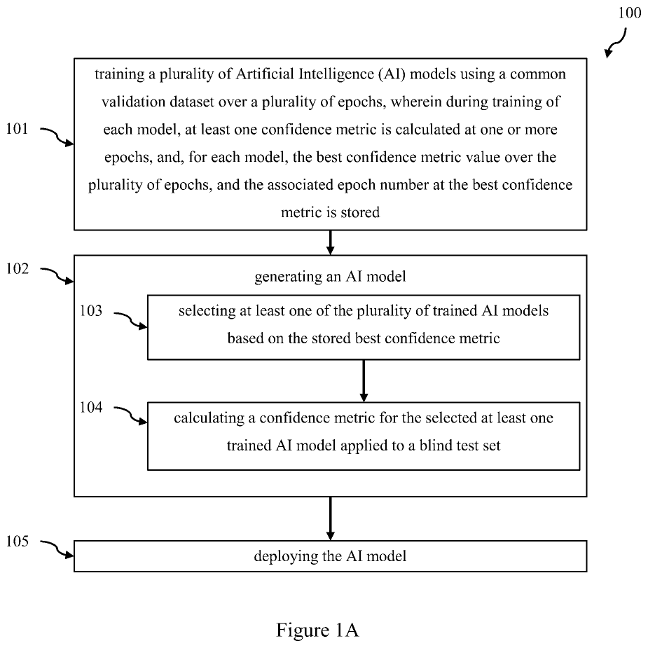

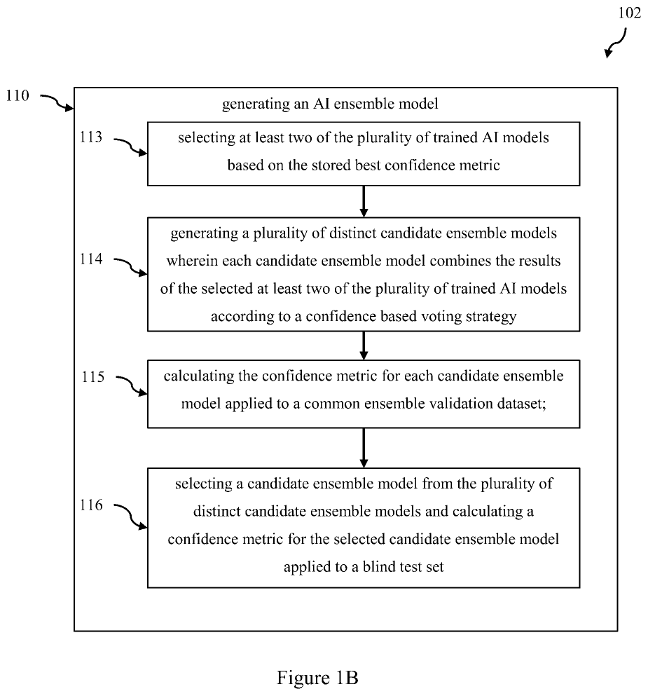

With reference to FIG. 1A, embodiments of methods for training AI models using metrics that take into account confidence, rather than just accuracy will now be discussed.

Most prior art AI training methods focus on total accuracy, or variations of total accuracy to judge the performance of an AI model. These may include the accuracy of the model on individual classes (of the categories of classification), i.e., ‘class accuracy,’ and accuracy variants, such as weighting the accuracy by the total number of images in each category or class, i.e., ‘balanced accuracy.’ However a problem with these accuracy focused metrics is that the translatability or generalizability of the AI model are not directly measured by these quantities.

The embodiments discussed herein can be used to create well-performing AI model that are guided by level of confidence (or distribution of the level of confidence/score) that the AI model can classify certain images/data correctly. Whilst accuracy may be calculated and used for final reporting, the methods incorporate one or more confidence metrics that measures this level of confidence correctly as an intermediate step in selecting the best AI model among many potential models, prior to reporting. As will be outlined below, using performance metrics (or simply metrics) that take into account confidence are more directly useful in establishing translatability of an AI model.

FIG. 1 is a schematic flowchart of the generation of an Artificial Intelligence (AI) model 100 according to an embodiment.

At 101 a plurality of Artificial Intelligence (AI) models are trained using a common validation dataset over a plurality of epochs. During training of each model at least one confidence metric is calculated over one or more epochs, and, for each model, the best confidence metric value over the plurality of epochs, and the associated epoch number at the best confidence is stored. Preferably the confidence metrics are calculated each epoch, or every few epochs.

At least one confidence metric may comprise a primary assessment metric, and one or more secondary assessment metrics. The secondary metrics may be used as tiebreaker metrics. In some embodiments at least one of the metrics is a confidence metric and at least one is an accuracy metric. The metrics may include accuracy, Mean class accuracy, sensitivity, specificity, a confusion matrix, Sensitivity-to-specificity ratio, precision, negative predictive value, balanced accuracy, Log loss, combined class Log loss, combined data-source Log loss, combined class and data-source Log loss, tangent score, bounded tangent score, per-class ratio of tangent score vs Log Loss, Sigmoid score, epoch number, mean of square error (MSE), root MSE, mean of average error, mean average precision (mAP), confidence score, Area-Under-the-Curve (AUC) threshold, Receiver Operating Characteristic (ROC) curve threshold, Precision-Recall curve. These metrics are discussed further below.

The plurality of AI models may comprise a plurality of distinct model configurations. Each model configuration comprises a model type (e.g., binary classification, multi-class classification, regression, object detection, etc.) and a model architecture or methodology (Machine Learning including Random Forest, Support Vector Machine, clustering; Deep Learning/Convolutional neural network including ResNet, DenseNet, or InceptionNet, including specific implementations such as a different number of layers and connections between layers, e.g., ResNet-18, ResNet-50, ResNet-101. We also extend the concept of distinct model configurations to include the use of distinct model inputs, hyper parameters, or pre-processing methods such as segmentation (where relevant). In one embodiment the AI models may comprise at least one AI model applied to unsegmented images and at least one AI model applied to segmented images.

The one or more pre-processing methods may comprise computer vision pre-processing methods to generate feature descriptors of an image. Computer vision models rely on identifying key features of the image and expressing them in terms of descriptors. These descriptors may encode qualities such as pixel variation, gray level, roughness of texture, fixed corner points or orientation of image gradients, which are implemented in the OpenCV or similar libraries. By selection on such feature to search for in each image, a model can be built by finding which arrangement of the features is a good indicator for a desired class (e.g., embryo viability). This procedure is best carried out by machine learning processes such as Random Forest or Support Vector Machines, which are able to separate the images in terms of their descriptions from the computer vision analysis.

Deep Learning and neural networks ‘learn’ features rather than relying on hand designed feature descriptors like machine learning models. This allows them to learn ‘feature representations’ that are tailored to the desired task. These methods are suitable for image analysis, as they are able to pick up both small details and overall morphological shapes in order to arrive at an overall classification. A variety of deep learning models are available each with different architectures (i.e., different number of layers and connections between layers) such as residual networks (e.g., ResNet-18, ResNet-50 and ResNet-101), densely connected networks (e.g., DenseNet-121 and DenseNet-161), and other variations (e.g., InceptionV4 and Inception-ResNetV2). Training involves trying different combinations of model parameters and hyper-parameters, including input image resolution, choice of optimizer, learning rate value and scheduling, momentum value, dropout, and initialization of the weights (pre-training). A loss function may be defined to assess performing of a model, and during training a Deep Learning model is optimized by varying learning rates to drive the update mechanism for the network's weight parameters to minimize an objective/loss function.

The plurality of trained AI models are then used to generate a final AI model 102. In one embodiment this comprises selecting at least one of the plurality of trained AI models based on the stored best confidence metric 103 and calculating a confidence metric for the selected at least one trained AI model applied to a blind test set 104. Generating the final AI model 102 may be performed using an ensemble method that uses at least two of the trained AI models based on the stored best confidence metrics and a confidence based voting strategy, a distillation method which uses at least two of the trained AI models to train a student model based using at least one confidence metrics, or some other selection method, such as by selecting at least two of the plurality of trained AI models, comparing each of at least two of the plurality of trained AI models using a confidence based metric, and then selecting the best trained AI models based on the comparison.

FIG. 1B is a flowchart of an ensemble model 110 for generating the final AI model 102. Two or more (including all) of the trained AI models are selected for inclusion in the ensemble model based on the confidence metrics 113. Each model is only considered once at its maximum performance, and multiple epochs of the same model are not included. To select the AI models for inclusion details may be ranked on a primary confidence metric. In one embodiment all models exceeding a threshold value are selected for inclusion in the ensemble model. In some embodiments other selection criteria in addition to the primary confidence metric may be used. For example secondary metrics (confidence based or accuracy based) and/or epoch numbers. Additionally or alternatively the models may be selected to ensure the AI models in the ensemble contain a range of different model architectures and computer vision pre-processing or segmentation techniques. That is when there are two models with similar model configurations (e.g., architecture) and similar primary metrics, only one is be selected as representative of that model configuration.

The selected AI models are used to generate a plurality of distinct candidate ensemble models 114. Each candidate ensemble model combines the results of the selected trained AI models according to a confidence based voting strategy to produce a single result.

The voting strategy defines the method by which the model scores are combined. In selecting ensembles, each voting strategy is considered part of the ensemble model, such that an ensemble model consists of:
the collection (or sub-collection) of AI models, and
the voting strategy.

The voting strategies may include confidence based strategies such as maximum confidence, mean confidence, majority-mean confidence, majority-max confidence, median confidence, weighted mean confidence, and other strategies that resolve the predictions from multiple models into a single score.

The confidence metric (and any secondary assessment metrics) are calculated for each candidate ensemble model applied to a common ensemble validation dataset 115. The common ensemble validation dataset may be the common validation dataset or an intermediate test set not used in training the plurality of Artificial Intelligence (AI) models (and distinct from the final blind test set). The best candidate ensemble model is selected based on the confidence metric 116 for the common ensemble validation dataset. Any secondary metrics may be used as tiebreakers between similar confidence metrics, or to assist in selecting the best model, e.g., if multiple metrics pass associated thresholds, wherein at least one of the multiple metrics is a confidence metric. Similarly, if for a first model the primary confidence metric is good, but the secondary metrics are poor, and for a second model we have a primary confidence metric that is also good, but less than the value for the first model, but the secondary metrics are also good, or at least much better than the secondary metrics for the first model, then we can select the second model.

The best candidate ensemble model is then applied to a blind test set (unchanged—that is with the same configuration and hyper-parameters) and we calculate the confidence metric and report. For example the report may include the distribution of scores associated with the final model, as well as a breakdown of individual datapoint, class, and data-source (i.e., for a medical application, breakdown of each patient, each class such as viable or non-viable embryo for IVF, and each clinic). This is an important consideration, as a well-generalizing model would be expected to have a high Accuracy metric on blind test sets, even if it was not selected using the metric of Accuracy. Selecting a model based on a confidence metric may indeed lead to improved performance in not only that metric, but also other metrics that are more commonly reported and understandable to people outside the field of AI, such as Accuracy.

We then deploy the AI ensemble model 105 for use on new datasets if the best confidence metric, e.g., the primary assessment metric, (on the blind test set) exceeds an acceptance threshold (e.g., 50%, 70%, 90%, 95%, etc.). If the model fails the threshold the process can be repeated with new training data or a different distribution of model configurations.

A model may be defined by its network weights and deployment may comprise exporting these network weights and loading them into a computational system (e.g., a cloud computing platform) to execute the final trained AI model 100 on new data. In some embodiments this may involve exporting or saving a checkpoint file or a model file using an appropriate function of the machine learning code/API. The checkpoint file may be a file generated by the machine learning code/library with a defined format which can be exported and then read back in (reloaded) using standard functions supplied as part of the machine learning code/API (e.g., ModelCheckpoint( ) and load_weights( )). The file format may directly sent or copied (e.g., ftp or similar protocols) or it be serialized and send using JSON, YAML or similar data transfer protocols. In some embodiments additional model metadata may be exported/saved and sent along with the network weights, such as model accuracy, number of epochs, etc., that may further characterize the model, or otherwise assist in constructing the model on another computational device (e.g., cloud platform, server or user computing device).

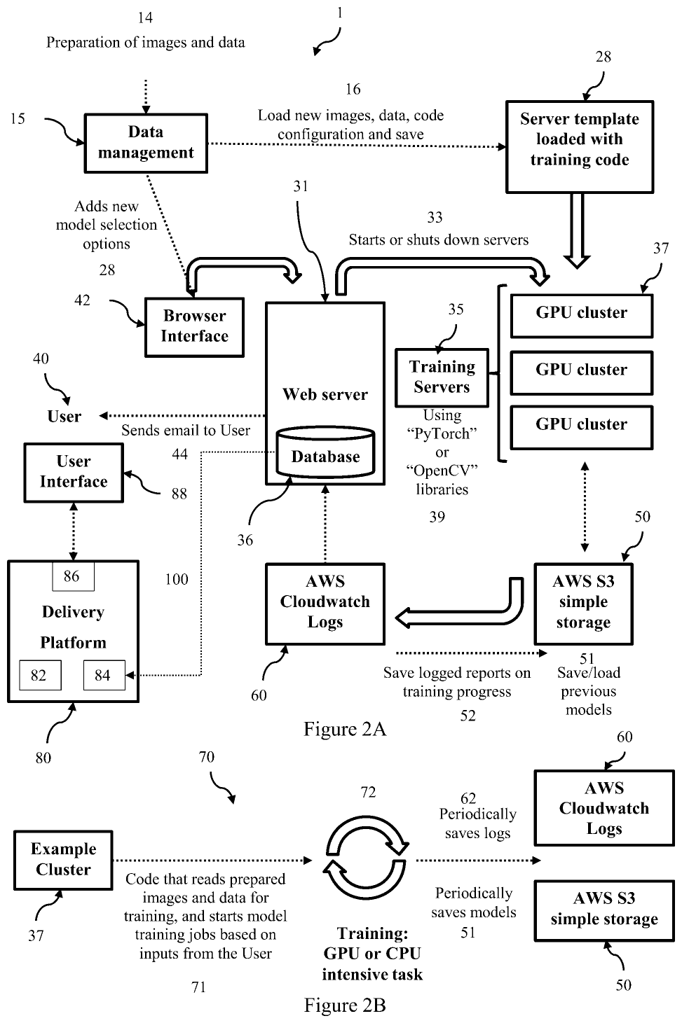

The computational generation of the AI model 100 can be further understood with reference to FIG. 2A which is a schematic architecture diagram of cloud based computation system 1 configured to generate and use an AI model 100 according to an embodiment. With reference to FIG. 1 the AI model generation method is handled by the model monitor 21.

The model monitor 21 requires a user 40 to provide data (including data items and/or images) and metadata 14 to a data management platform which includes a data repository. A data preparation step is performed, for example to move the data items or image to a specific folder, and to rename and perform pre-processing on any images such as objection detection, segmentation, alpha channel removal, padding, cropping/localizing, normalizing, scaling, etc. Feature descriptors may also be calculated, and augmented images generated in advance. However additional pre-processing including augmentation may also be performed during training (i.e., on the fly). Images may also undergo quality assessment, to allow rejection of clearly poor images and allow capture of replacement images. The data such as patient records or other clinical data is processed (prepared) to extract a classification outcome such as viable or non-viable in binary classification, an output class in a multi-class classification, or other outcome measure in non-classification cases, which is linked or associated with each image or data item to enable use in training the AI models and/or in assessment. The prepared data is loaded 16 onto a cloud provider (e.g., AWS) template server 28 with the most recent version of the training algorithms. The template server is saved, and multiple copies made across a range of training server clusters 37 (which may be CPU, GPU, ASIC, FPGA, or TPU (Tensor Processing Unit)-based) which form training servers 35.

The model monitor web server 31 then applies for a training server 37 from a plurality of cloud based training servers 35 for each job submitted by the user 40. Each training server 35 runs the pre-prepared code (from template server 28) for training an AI model, using a library such as Pytorch, Tensorflow or equivalent, and may use a computer vision library such as OpenCV. PyTorch and OpenCV are open-source libraries with low-level commands for constructing CV machine learning models. The AI models may be deep learning models or machine learning models, including CV based machine learning models.

The training servers 37 manage the training process. This may include dividing the data or images in to training, validation, and blind validation sets, for example using a random allocation process. Further during a training-validation cycle the training servers 37 may also randomize the set of images at the start of the cycle so that each cycle a different subset of images are analyzed, or are analyzed in a different ordering. If pre-processing was not performed earlier or was incomplete (e.g., during data management) then additional pre-processing may be performed including object detection, segmentation and generation of masked data sets, calculation/estimation of CV feature descriptors, and generating data augmentations. Pre-processing may also include padding, normalizing, etc., of images as required. Similar processes may be performed on non-image data. That is the pre-processing may be performed prior to training, during training, or some combination (i.e., distributed pre-processing). The number of training servers 35 being run can be managed from the browser interface. As the training progresses, logging information about the status of the training is recorded 62 onto a distributed logging service such as CloudWatch 60. Metrics are calculated and information is also parsed out of the logs and saved into a relational database 36. The models are also periodically saved 51 to a data storage (e.g., AWS Simple Storage Service (S3) or similar cloud storage service) 50 so they can be retrieved and loaded at a later date (for example to restart in case of an error or other stoppage). The user 40 is sent email updates 44 regarding the status of the training servers if their jobs are complete, or an error is encountered.

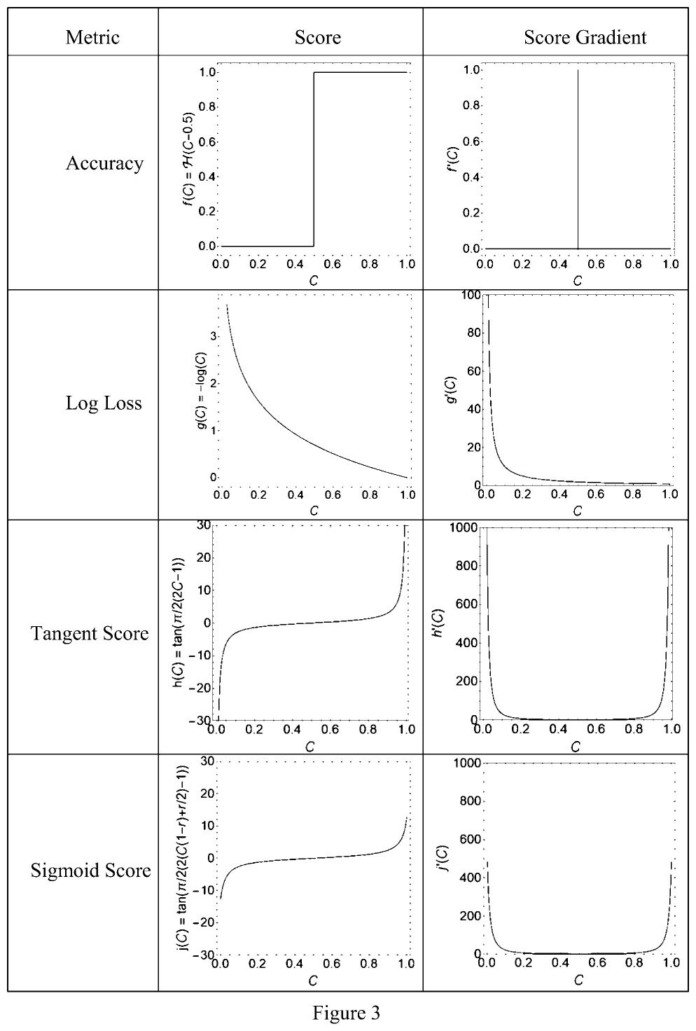

Within each training cluster 37, a number of processes take place. Once a cluster is started via the web server 31, a script is automatically run, which reads the prepared images and patient records, and begins the specific Pytorch/OpenCV training code requested 71. The input parameters for the model training 28 are supplied by the user 40 via the browser interface 42 or via a configuration script. The training process 72 is then initiated for the requested model parameters, and can be a lengthy and intensive task. Therefore, so as not to lose progress while the training is in progress, the logs are periodically saved 62 to the logging (e.g., AWS CloudWatch) service 60, and the current version of the model (while training) is saved 51 to the data (e.g., S3) storage service 51 for later retrieval and use. An embodiment of a schematic flowchart of a model training process on a training server is shown in FIG. 3B. With access to a range of trained AI models on the data storage service, multiple models can be combined together for example using ensemble, distillation or similar approaches in order to incorporate a range of deep learning models (e.g., PyTorch) and/or targeted computer vision models (e.g., OpenCV) to generate a robust AI model 100 which is then deployed to a delivery platform 80. As outlined above a model may be defined by its network weights and deployment may comprise exporting these network weights and loading them onto the delivery platform 80 to execute the final trained AI model 100 on new data. The delivery platform may be a cloud based computational system, a server based computational system, or other computational system, and the same computational system used to train the AI model may be used to deploy the AI model. In some embodiments the same computational system used to train the AI model may be used to deploy the AI model, and thus deployment comprises storing the trained AI model, for example in a memory of webserver 31, or exporting the model weights for loading onto a delivery server.

The delivery platform 80 is a computational system comprising one or more processors 82, one or more memories 84, and a communications interface 86. The memories 84 are configured to store the trained AI model, which may be received from the model monitor web server 31 via the communications interface 86 or loaded from an export of the model stored on an electronic storage device. The processors 82 are configured to receive input data via the communications interface (e.g., an image for classification from user 40) and process the input data using the stored AI model to generate a model result (eg a classification), and the communications interface 84 is configured to send or the model result to a user interface 88 or export to a data storage device or electronic report. the processors are configured to receive input data and process the input data using the stored trained AI model to generate a model result. A communications module 86 is configured to receive the input data and send or store the model result. The communications module may communicate with a user interface 88, such as a web application to receive the input data and to display the model result, e.g., a classification, object bounding box, segmentation boundary, etc. The user interface 88 may be executed on a user computing device and is configured to allow user(s) 40 to drag and drop data or images directly onto the user interface (or other local application) 88, which triggers the system to perform any pre-processing (if required) of the data or image and passes the data or image to the trained/validated AI model 100 to obtain a classification or model result (e.g., objecting bounding box, segmentation boundary, etc.) which can be immediately returned to the user in a report and/or displayed in the user interface 88. The user interface (or local application) 88 also allows users to store data such as images and patient information in data storage device such as a database, create a variety of reports on the data, create audit reports on the usage of the tool for their organization, group or specific users, as well as billing and user accounts (e.g., create users, delete users, reset passwords, change access levels, etc.). The delivery platform 30 may be cloud based and may also enable product admin to access the system to create new customer accounts and users, reset passwords, as well as access to customer/user accounts (including data and screens) to facilitate technical support.

A range of metrics may be used for the primary and secondary assessment metrics. Accuracy based metrics include accuracy, mean class accuracy, sensitivity, specificity, a confusion matrix, Sensitivity-to-specificity ratio, precision, negative predictive value, and balanced accuracy, typically used for classification model types, as well as mean of square error (MSE), root MSE, mean of average error, mean average precision (mAP) typically used for regression and object detection model types.

Confidence based metrics include Log loss, combined class Log loss, combined data-source Log loss, combined class and data-source Log loss, tangent score, bounded tangent score, per-class ratio of tangent score vs Log Loss, Sigmoid score. Other metrics include epoch number, Area-Under-the-Curve (AUC) thresholds, Receiver Operating Characteristic (ROC) curve thresholds, and Precision-Recall curves which are indicative of stability and transferability.

These metrics are discussed further below. However it is to be understood that these are representative only, and variations and other accuracy or confidence based metrics, may be used.

Accuracy

This metric is defined as the total number of correctly identified data (regardless of class) divided by the total number of data in the set on which the accuracy is quoted. This is typically a validation set, blind test set or double-blind test set. This is the most common metric quoted in the literature, and is appropriate for very large and well-curated datasets, but suffers from being a poorer measure for translatability for real industry datasets, especially if the data is sourced from a different distribution than the original training and validation sets. Accuracy also suffers as a metric when a model is applied to a highly unbalanced class distribution, i.e., in some cases, with a strong majority and minority class, high accuracy can be achieved simply by predicting only the majority class.

Mean Class Accuracy

This metric is defined as simply the sum of the percentage accuracies of each class, divided by the total number of classes. Since each class accuracy is expressed as a percentage, a model that performs well on overall accuracy on an uneven dataset (e.g., most of the data is one class only, such as most embryo images being viable in an embryo dataset, and the model being biased towards that class), will nevertheless not score highly on this metric. This provides a quick assessment as to whether the model is getting many examples right across each class. It is often very similar in its performance, in practice, to the Balanced accuracy below, especially in cases where the total number of examples within each class, in the validation or test sets, is similar. For highly unbalanced sample datasets, reporting the mean class accuracy can nevertheless still be misleading, as it will heavily favor models that performed well on the smaller class (i.e., in situations where models performed uncharacteristically well or poorly on the smaller class, which has larger statistical fluctuations associated with its smaller amount of data).

Sensitivity or Recall (true positive rate—TPR)

Sensitivity, TPR and Recall are synonyms, and takes the form:

TPR=TP/(TP+FN), Equation 1

where TP is the total number of true positive examples on the set being measured (the prediction was positive and the outcome was positive), and FN is the total number of false negatives on the set being measured (the prediction was negative and the outcome was positive).

This quantity represents the ability of the model to detect ‘positive’ examples of the classification on which it was trained, e.g., embryo viability, PGT-A aneuploidy, or the detection of a cancer. What constitutes a positive example or class is dependent on the classification problem that the model has been trained on, and different industry problems will exhibit different levels of usefulness in focusing on the metric of Sensitivity or Recall. In some cases it can represent a more reliable indicator of a well-translating model, but only in circumstances where the model was not too unbalanced, or wildly varying in its class accuracy, and in cases where the sensitivity is less amenable to label noise, such as the case of embryo viability (where label noise is more dominant in the non-viable embryo class). As an example, in cases where a model would classify viable embryos at a high rate (>90%) and non-viable embryos at a low rate (<20%) it is a poor indicator of translatability. It is therefore useful for this metric to be combined with other metrics together. In the embryo binary classification example above, this would ensure that the model is not a) reducing in accuracy on non-viable embryos, orb) luckily landing on a very easily classified set of viable embryos at a specific epoch, which is misleading for that overall model performance.

Specificity (True Negative Rate—TNR)

Specificity or TNR takes the form:

TNR=TN/(TN+FP) Equation 2

where TN is the total number of true negative examples on the set being measured (the prediction was negative and the outcome was negative), and FP is the total number of false positives on the set being measured (the prediction was positive and the outcome was negative).

This quantity represents the ability of the model to detect ‘negative’ examples of the classification on which it was trained. In the case of binary classification models, the sensitivity and the specificity are the only two class-specific accuracies available. The class accuracies of all classes are important to examine across the full set and also the breakdown of the individual and separate data-sources. In the case of the embryo viability problem above, it is important to look at the non-viable accuracy, not only for the total test set, but also for the separate clinic breakdown of the full test set. In the case of embryo non-invasive PGT-A models, specificity relates to the euploid class of embryos, and in the case of cancer detection, relates to the non-cancerous samples.

Confusion Matrix

The confusion matrix is simply a tabular representation of the four quantities defined above: the total number of true positives (TP), true negatives (TN), false positives (FP) and false negatives (FN). Note that the calculation of the confusion matrix and each of the four quantities requires a threshold to be established. This is the value above which outputs from the model (i.e., the predicted score) will be considered positive, and below which will be considered negative. For a binary classification problem, such as embryo viability classification, it is common to train models so that the threshold is set to 50% out of 100% (i.e., normalized, and equal weighting between the two classes), however this does not need to be the case. In the case of Ensemble models, the total combined ensemble model may have a threshold that is different from the individual models that comprise it. In order to establish the best performing threshold, this procedure should be carried out on a validation set to avoid over-fitting the test set. The method for assessing the threshold involves scanning over all possible threshold values, which can take the form of an Area-Under-the-Curve (AUC) or Receiver Operating Characteristic (ROC) curve, or a Precision-Recall (PR) curve. This metric is described below.

Sensitivity-to-Specificity Ratio

While data sourced from some localities can be more difficult to stabilize, attempting to have even accuracies across the classes and the different localities at the same time represents competing effects that can be difficult to attain. In some cases, the ratios among the class accuracies may preferentially be unequal, especially if noise or other bad data is not equally distributed among the classes to be classified. In the case of embryo viability classification, the ratio of sensitivity to specificity has been shown to be greater than 1 when translating optimally. Therefore, a combination metric, Sensitivity-to-specificity ratio can be defined as sensitivity/specificity, and is a useful metric, but its best value is dependent on the problem to be solved.

Precision (Positive Predictive Value—PPV)

PPV takes the form:

PPV=TP/(TP+FP) Equation 3

This quantity represents the percentage of total positive predictions that were correctly classified. It is often used in conjunction with Recall as a way of characterizing the performance of a model in a way that is less vulnerable to bias on strongly unbalanced datasets (see Graphical information below). It can be calculated directly from the confusion matrix.

Negative Predictive Value—NPV

NPV takes the form:

PPV=TN/(TN+FN) Equation 4

This quantity represents the percentage of total negative predictions that were correctly classified, and is the counterpart to PPV. It can be calculated directly from the confusion matrix.

F1-Score:

The F1-score, is defined as:

2*Precision*Recall/(Precision+Recall) Equation 5

This metric provides a combined metric between precision and recall that is less vulnerable to highly unbalanced datasets.

Balanced Accuracy:

The balanced accuracy is defined as:

(Sensitivity+Specificity)/2 Equation 6

This metric is an overall accuracy metric, as an alternative to Accuracy as defined above, giving equal weight to specificity and sensitivity.

(Negative) Log Loss

Log Loss of a classification model where the prediction is a value between 0 and 1 is defined as:

−log( [symbol] ) Equation 7

where [symbol] =1−|y_{prediction}−y_{target}| offers a measure of the level of “correctness” of the prediction, where [symbol] =1 means the prediction is perfectly matches the target label and [symbol] =0 means the prediction is completely opposite to the target label.

Log Loss is the most direct measure of model performance with regard to itself, as it is related to the cross-entropy loss function that is used to optimize the model itself during training. It measures the performance of a classification model where the prediction is a value between 0 and 1. Therefore, Log Loss inherently takes into account the uncertainty of the predicted score based on how much it deviates from the correct classification. Log Loss is a class of confidence metric.

Confidence metrics take into consideration: (1) for each datapoint the confidence in predicting that class, which is the distance in the distribution between the score for correct classification (which should be higher) and incorrect classifications (which should be lower); and (2) across all classes the confidence in predicting each class, which is ensuring a balanced and high distribution of confidence scores between the classes.

In practice, analysis of models that perform well according to confidence metrics have some correlation with those chosen based on an Accuracy (or Balanced Accuracy or Mean Class Accuracy) metric. Confidence metrics will tend to favor higher-epoch results but will often produces similar per-epoch behavior compared with other metrics. This makes sense, as it is selecting models that perform with an AI score distribution that is highly separated, i.e., there is a clear distinction between correct and incorrect predictions. This doesn't of itself mean the model will work well for images that have unexpected features (resolution, color balance) or that the model will behave stably across the breakdown of data-sources that make up the full dataset. However, it is an indicator that at a specific epoch the model generalized well.

An important aspect of selecting a stable model is also that the model loss (or other metric) is consistent across multiple epochs and remains stable (or until an over-trained point). To uncover this, the graphical (per-epoch) information may be considered.

Log Loss for Individual Classes (and Combined): Combined Class Log Loss

We propose that Log Loss may also be computed for separate classes individually, which can provide distribution information for each category. This is useful in cases where the classes are unbalanced or contain different amounts of noise from each other. In these cases, Log Loss on one class may provide a better indication of generalization than that of another class. In general, Log Loss associated with a less noisy class will provide the best measure of generalization.

The Log Loss for individual classes can then be summed to give a Combined Class Log Loss, which differs from the total Log Loss (as it gives equal weight to each class regardless of the total number of samples represented in each class).

Log Loss for Individual Data-Sources (and Combined): Combined Data-Source Log Loss

We propose that Log Loss may also be computed for separate data-sources individually, which can provide distribution information for each data-source and ensure that the selected model is generalizing well across different (and likely diverse) data-sources, and not biased to an individual or sub-set of data-sources. This can be a good measure of AI generalization.

This is also useful in cases where the size of data between data-sources is unbalanced or sources contain different amounts of noise from each other. In these cases, Log Loss on one data-source may provide a better indication of generalization than that of another class. In general, Log Loss associated with a less noisy data-source will provide the best measure of generalization.

The Log Loss for individual data-sources can then be summed to give a Combined Data-Source Log Loss, which differs from the total Log Loss (as it gives equal weight to each data-source regardless of the total number of samples represented in each data-source).

Log Loss for Individual Classes and Data-Sources (and Combined): Combined Class and Data-Source Log Loss

We propose that Combined Class Log Loss and Combined Data-Source Log Loss described above be combined to ensure maximum generalizability, considering both generalization across classes and (different and diverse) data-sources.

The Log Loss for individual classes and data-sources can then be summed to give a Combined Class and Data-Source Log Loss, which differs from the total Log Loss (as it gives equal weight to each data-source regardless of the total number of samples represented in each class and data-source).

Tangent Score

Tangent Score of a classification model where the prediction is a value between 0 and 1 is defined as:

$$tan \frac{π}{2} ( 2 - 1 ) \qquad Equation 8$$

Bounded Tangent Score

One practical adjustment to the tangent score function is to rescale

$$∈ [ \frac{r}{2} , 1 - \frac{r}{2} ] ,$$

such that the metric is bounded, avoiding run-off scores of ±∞ as [symbol] →0 or [symbol] →1, defined as:

$$tan \frac{π}{2} ( 2 ( ( 1 - r ) + \frac{r}{2} ) - 1 ) \qquad Equation 9$$

where 0<r<1, but is typically chosen to be a small number (e.g., r=0.05)

Tangent Score is used to offset the undesirable tendency of Log Loss, which disproportionately “punishes” model predictions that are confidently incorrect, by rewarding model predictions that are confidently correct. An upper and lower bound can be used to clip the tangent score when the argument is near asymptotes

$$x = \pm \frac{π}{2} ( where tan ( x ) \to \pm ∞ ) .$$

Per-Class Ratio of Tangent Score Vs Log Loss

When a binary dataset contains incorrect labels in one class, the Per-Class Ratio of Tangent Score vs Log Loss metric can balance the undesirable effects of both Log Loss (which unfairly punishes a model trained on poor quality data) and tangent score (which can result in high rates of false confident predictions in the clean class).

We propose that calculating the ratio between Tangent Score on the unclean class (class with significant label error rates) and Log Loss on the clean class (class with negligible label error rates), provides a metric that can offset the deleterious effects of either individual metrics. This situation only applies to cases where one class has a distinctly higher level of label error rates.

Using Per-Class Ratio of Tangent Score vs Log Loss as the primary metric, FIGS. 3A and 3C represent the histograms of the ratios, from 0.0 to 1.0, with a binary threshold of 0.5, for viable embryos (indicated by vertical dashed line). Correctly classified embryos are shown as bars with thick horizontal lines (True Positives) 32, and incorrectly classified embryos are shown as black columns (False Negatives) 31. FIGS. 3B and 3D show the equivalent histograms for the non-viable embryos, where correctly classified embryos are shown as bars with horizontal lines (True Negatives) 34, and incorrectly classified embryos are shown as bars with thick rearward diagonal lines (False Positives) 33.

Sigmoid Score

Sigmoid Score of a classification model where the prediction is a value between 0 and 1 is defined as:

$$\frac{2}{1 + {e}^{- k ( 2 - 1 )}} - 1 \qquad Equation 9$$

where k is a decay constant.

Sigmoid Score is a “soft” alternative to other Accuracy metrics, in that provides a graded measurement of model performance rather than a sharp cut-off.

Score Gradients (a.k.a. Marginal Sensitivity):

FIG. 3 show the Score and Score gradient for the metrics Accuracy, Log Loss, Tangent Score and Sigmoid Score with respect to C, which illustrates the marginal sensitivities of the various metrics. Depending upon the specific problem and underlying data distribution (or suspected distribution), an appropriate confidence based metric can be selected (i.e., that best suits the data).

A range of other model selection criteria may also be used.

Epoch Number

A very coarse measure of the performance of a model during training is the number of passes (or epochs) through the training set it has achieved. While this information does not provide the richer analytics and insights into the balanced between classes, or distributions of the predicted scores obtained from the model that the other metrics can provide, it nevertheless provides high-level information about the model, namely, a sense as to whether the model has converged, i.e., whether the model has reached a steady state where no improvement is likely to occur by continuing training the model. This is related to the graphical representation of the loss, on the training set and the validation set, which is described more fully below. Furthermore, models trained to higher epochs are also more likely to have been exposed to all available data augmentations available in the training process, and are also more likely to be confident in the predictions (i.e., their distribution of predicted scores will contain more high-confidence examples). A model trained to an extremely high epoch may also exhibit loss of generality due to over-training. Therefore, this metric is only to be used as a very coarse measure.

Metrics for Non-Classification Models

While the metrics obtained from the confusion matrix and other related Accuracy measures are usually used for (binary) classification problems, other types of models exist, which can use different metrics. Some of these other metrics are: Mean of square error (MSE), root MSE, mean of average error, mean average precision (mAP), and confidence score, which are used in regression and object detection models.

Graphical Information

Graphical information regarding the training process which has taken place, such as plots describing the loss as a function of epoch (for both the training set and the validation set), is instructive for determining whether the model a) has systematically improved its loss over a range of epochs and thus learned information, b) has converged to a steady state, and c) has not overtrained (i.e., the validation loss deteriorates while the training loss continues to improve).

The distribution of the scores at each epoch, displayed as a histogram or other plot style for visualizing the distribution, can provide an indication of the model performance. For example, if the distribution of prediction scores from a model attempting to solve a binary classification problem is bi-modal, and the modes are well-separated, this is an indicator of translatability. If however, the distribution is Gaussian-like, then the chance of correct classification being higher than incorrect classification is likely to be brittle, as the majority of scores are clustered around the decision threshold and likely no better than random chance, and thus unlikely to generalize well to an unseen dataset.

Area-Under-the-Curve (AUC) or Receiver Operating Characteristic (ROC) curves are common visualization tools for identifying the decision threshold of a model, i.e., the threshold above which a prediction score is considered to be a prediction of viability, and below which is considered to be a prediction of non-viability (in the case of a binary classification problem). It is created by plotting the TPR against the FPR. ROC curves are also useful for visually assessing whether the best threshold for a given model has significant predictive power compared to random chance. However, they can also be considered unreliable in the case of highly unbalanced datasets.

Precision-Recall curves are often recommended for strongly unbalanced datasets. This is due to the avoidance of the total number of true negatives in the calculation of either the Recall or the Precision. For example, as the ratio of the number of negative outcome data to positive outcome data changes, the Precision-Recall curve should remain roughly invariant.

To further illustrate the method, we will now consider application to the development of an embryo viability binary classification model for selecting Embryos for implantation in an IVF procedure. The dataset comprises 2D static optical light microscope images of Day 5 blastocyst embryo. Three case studies using different metrics will now be presented. A range of top-performing models are obtained based on a primary accuracy and/or other metrics, and compared with top-performing models based on the confidence metrics. The models are then applied to blind test sets reserved for these experimental comparisons, to assess if there is a difference in the robustness/generalizability of the model when it is transferred to a new dataset. Whether a metric should be used alone, with other metrics, or at all, was also empirically explored/tested.

We begin by first comparing a variety of metrics in terms of their generalization and consistency across multiple epochs. Then, the focus will be on selection of models with respect to the preferred metric for this problem: Log Loss.

Additional secondary measures that are relevant to this problem include:
Balanced accuracy;
Sensitivity-to-Specificity ratio;
Log Loss for individual classes—(e.g., non-viable and viable);
Epoch number; and
Any unused primary metrics of confidence scores

In the case of Sensitivity-to-Specificity ratio, a range of models should be selected such that this metric varies across the models to be ensembled, in order to provide a robust ensemble that includes models with different biases toward different sub-populations of embryos.

In the case of epoch number, it is intended to avoid models during training that performed well according to the primary metric due to chance, without adequate time to make full use of training methods (e.g., augmentations) that require many epochs. Therefore, a minimum number of epochs are specified to screen these cases from contributing to the ensemble (i.e., a minimum epoch threshold).

The dataset for these embodiments comprises 3,987 images from 7 separate clinical regions, comprising 11 sites in total. Viability was assessed based on detection of fetal heartbeat at the first ultrasound scan after implantation (typically 6-8 weeks).

For simplicity, the names of clinic-datasets are denoted as clinic-data 1, clinic-data 2 and so forth. Table 1 summarizes the class size (total number of non-viable or viable images) and total size of 7 clinic-datasets, where it can be seen that class distributions vary significantly between datasets. In total, there are 3,987 images for model training and evaluation purposes.

TABLE 1
Dataset description.

| | Class | Class | |
|---|---|---|---|
| | non-viable | viable | Total |
| Sub-dataset | size | size | size |
| Clinic-data 1 | 106 | 180 | 286 |
| Clinic-data 2 | 335 | 317 | 652 |
| Clinic-data 3 | 129 | 202 | 331 |
| Clinic-data 4 | 191 | 218 | 409 |
| Clinic-data 5 | 491 | 475 | 966 |
| Clinic-data 6 | 780 | 337 | 1117 |
| Clinic-data 7 | 121 | 105 | 226 |
| Sub-dataset | Class non-viable size | Class viable size | Total size |
| Total | 2153 | 1834 | 3987 |

Comparison of Metrics, Generalisation and Consistency.

The selection of models according to a specific metric, as measured on a validation set, can be assessed by examining the consistency of the specific selection metric across the validation and test sets, the generalization of the model with respect to the Balanced Accuracy (i.e., does the model accuracy generalize well for a given selection metric, which may not be Balanced Accuracy), and the distribution of the scores as displayed by a histogram.

In Table 2 below, results for Balanced Accuracy values are presented for several trained AI models, each selected from a large cohort of models with distinct model configurations including different training parameters and using different primary selection metrics. It is found that AI models using Mean Class Accuracy, and Balanced Accuracy as the primary metric typically arrive at a similar trained AI model and epoch. While the Balanced Accuracy on the validation set is high for this problem (67.6%), it drops significantly for the test set (58%), indicating that the model, while translating to the validation set, is not generalizing well to the test (blind) datasets (which include double-blind datasets, that is, data from separate data-sources in which none of the data was used in training), and it is by no means certain that these metrics are the best to use for model selection.

In the case of Log Loss as the selection metric (a confidence based metric), the Balanced Accuracy on the validation set is less than that of the Accuracy metrics, however, there is an improvement in the Balanced Accuracy as measured on the test set. Further investigation into the Log Loss below, a confidence metric, will reveal that the metric is the most reliable for generalization and thus model selection. Recall presents a reversed issue, where an under-performing model with respect to Balanced Accuracy on the validation set can perform significantly better on Balanced Accuracy on the test set. This particular feature is specific to the embryo viability problem, where Recall (or classifying viable embryos) represents a less label-noise heavy dataset, whereas non-viable embryo datasets contain significantly greater label noise. While the focus here is on the efficacy of the selection metric, Recall (which effectively ignores the non-viable accuracy) cannot be used alone as selection metric, as it is vulnerable to models that classify positive examples at 100% accuracy, but low accuracy at negative examples. Nevertheless, Recall represents an important selection metric which is, for this problem, important to consider as a main selection metric. Precision, on the other hand, performs similarly as a selection metric as other Accuracy measures.

TABLE 2
A comparison of the Balanced Accuracy metric on a validation
set and a combined blind-double-blind test set, for
a variety of selection metrics. The epoch number associated
with the selected model is also shown

| | | Validation | Test |
|---|---|---|---|
| | | Balanced | Balanced |
| Epoch | Selection metric | Accuracy | Accuracy |
| 77 | Mean Class Accuracy and | 67.63829787 | 58.14548372 |
| | Balanced Accuracy | | |
| 38 | Log Loss | 67.10638298 | 61.40328533 |
| 81 | Recall | 61.7786391 | 66.05088945 |
| 77 | Precision | 67.63829787 | 58.14548372 |

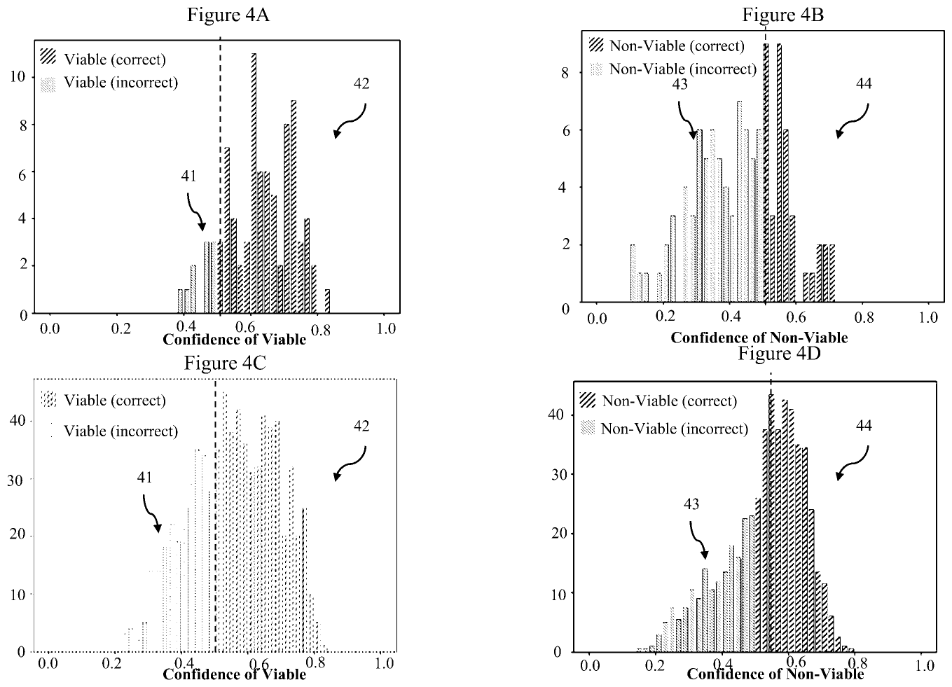

Using Recall as a primary selection metric, the distribution of the scores extracted from the classification model are examined, in FIGS. 4A and 4B for the validation set, and FIGS. 4C and 4D for the test set. FIGS. 4A and 4C represent the histograms of scores, from 0.0 to 1.0, with a binary threshold of 0.5, for viable embryos (indicated by vertical dashed line). Correctly classified embryos are colored as bars with thick forward diagonal lines (True Positives) 42, and incorrectly classified embryos are colored as bars with thin rearward diagonal lines (False Negatives) 41. FIGS. 4B and 4D show the equivalent histograms for the non-viable embryos, where correctly classified embryos are colored as bars with thick forward diagonal lines (True Negatives) 44, and incorrectly classified embryos are colored as bars with thin rearward diagonal lines (False Positives) 43.

Note that the test set contains a distribution of clinics, containing both blind and double-blind test examples (where double-blind data have been sourced from clinics that are not represented in the training or validation sets, and thus the distribution of data will be different). While the performance on the model is skewed towards viable embryos on the validation set, an inherent property of focusing on Recall as a selection metric, a comparison of FIGS. 4A and 4B shows that the distribution of scores on the test set is not well-defined. With a single Gaussian-like (mono-modal) distribution around the threshold value of 0.5, the high performance of the model with respect to Balanced Accuracy is more likely to be based on chance, and unlikely to generalize well to a new double-blind set.

A similar comparison can be made between FIGS. 4B and 4D, where a distribution on the validation set, which is not well-separated, remains poorly separated on a test set, and so is unlikely to provide strong generalization.

Metrics for Ensemble Model with Constituent AI Models Chosen Based on Balanced Accuracy.

In this section trained AI models are selected for inclusion into an Ensemble based on the Balanced Accuracy on a shared validation as a primary metric. The best performing models (based on Balanced Accuracy) were selected, and a voting strategy of Majority-Mean confidence used to combine candidate ensembles. A breakdown of the model performance associated with these metrics by class is also considered.

A shared validation set of 252 images is considered, in which the Ensemble model constituents were chosen. The model was then applied to a blind test set of 527 images for comparison.

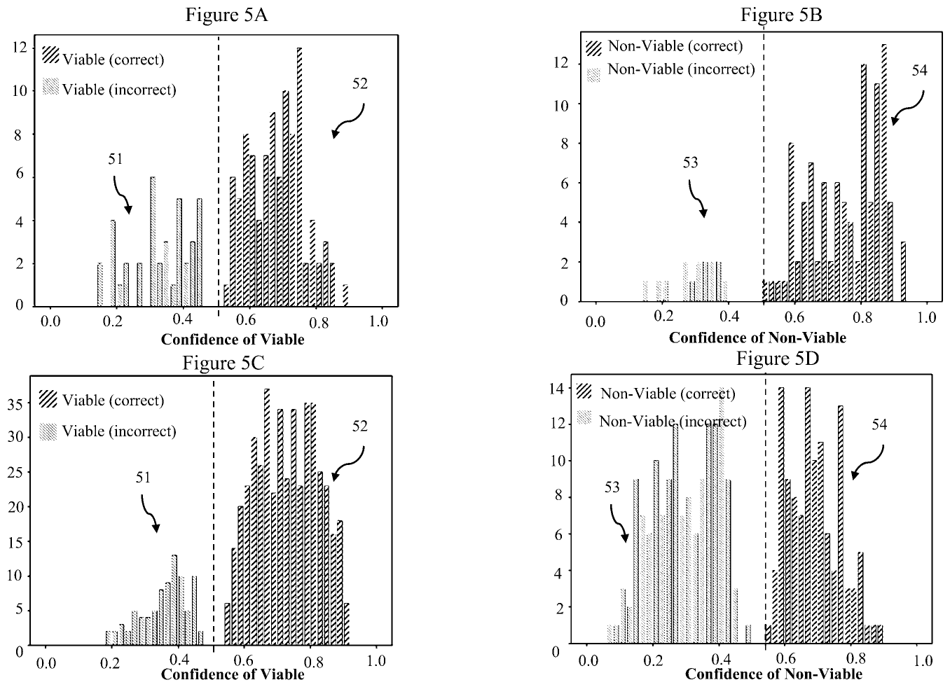

The histogram associated with the scores assigned by the ensemble model to the viable embryos on the shared validation set can be seen in FIG. 5A where correctly classified embryos are colored as bars with thick forward diagonal lines (True Positives) 52, and incorrectly classified embryos are colored as bars with thin rearward diagonal lines (False Negatives) 51. The equivalent histogram for non-viable embryos is shown in FIG. 5B where correctly classified embryos are colored as bars with thick forward diagonal lines (True Negatives) 54, and incorrectly classified embryos are colored as bars with thin rearward diagonal lines (False Positives) 53. The model distribution is better separated than the single-model case above, due to the fact that Ensemble models generally exhibit improved performance in both Accuracy measures and generalization compared to single models. This is because multiple models vote on a single image, allowing greater scope for model discrepancies to be outvoted or handled by the wider scope of attention preferences of the constituent models. The details of the bias among the models are related to the voting strategy among the models, which, together with the constituents themselves, defines the Ensemble model.

Note that the histograms associated with the results on the validation set, in FIGS. 5C and 5D, show good separation between the correctly 52, 54 and incorrectly identified embryos 51, 53 (bi-modal distribution), and both exhibit a high value of Accuracy as measured by TPR and TNR, discussed below in the section on Class Breakdown. The separation between the correctly and incorrectly identified embryos on the blind test set persists, which is a sign of generalization. However, the value of accuracy drops, as exhibited in FIG. 5D, where a large number of False Positives is observed, which decreases the value of the Specificity. This is an inherent problem of noisy datasets, where high noise in non-viable embryo datasets contributes to the reduction of generalization.

Note the importance of the quality of the dataset (e.g., label quality or correctness) in exhibiting generalization, and how the choice of metric in the selection of an Ensemble model constituents, and the selection of the model out of a group of Ensemble models, is a large contributing factor in the ultimate generalization or translatability of a model. A breakdown of metrics measuring the performance of the model on the validation and test sets is presented in the next section.

Class Breakdown

Metrics associated with the breakdown of results for the two classes, viable and non-viable examples, are shown in Table 3, for all clinics represented in the combined validation set. While the Accuracy measures are high for both classes, and establish the benchmark for the associated Log Loss values, Table 4 shows a drop in the Accuracy for ‘Class 0,’ or non-viable embryos, as expected due to label noise, when applied to the blind test set. However, the Accuracy for ‘Class 1’ or viable embryos, remains high. Note, however, that the Log Loss deteriorates due to the reduction in the non-viable accuracy, or Specificity, but the distribution associated with FIG. 5D is still well-separated, and since Log Loss takes into account the score distribution information, the Log Loss is a more reliable metric for AI generalization.

The class-specific Log Losses are also compared and combined, and it is found that the selection of models based on these metrics are consistent with Log Loss.

TABLE 3
The class breakdown of the Ensemble model with candidate AI models chosen based on Balanced
Accuracy is shown on a shared validation set including the mean, balanced and combined class
metrics.

| | | | Class 0 | Class 1 | | |
|---|---|---|---|---|---|---|
| | Mean class | Balanced | Accuracy | Accuracy | | |
| Clinic | accuracy | Accuracy | (TPR) | (TNR) | F1-Score | Log Loss |
| Combined | 78.96825 | 79.51567 | 87.17949 | 71.85185 | 0.7951567 | 0.51441592 |

TABLE 4
The class breakdown of the Ensemble model Metrics for Ensemble model with candidate AI models
chosen based on Balanced Accuracy is shown on a shared validation set including the mean,
balanced and combined class metrics.

| | | | Class 0 | Class 1 | | |
|---|---|---|---|---|---|---|
| | Mean class | Balanced | Accuracy | Accuracy | | |
| Clinic | accuracy | Accuracy | (TPR) | (TNR) | F1-Score | Log Loss |
| Combined | 73.2447818 | 70.3596615 | 58.6358247 | 82.0834983 | 0.70359661 | 0.5529281 |

Metrics for an Ensemble Model Chosen Based on Log Loss as the Primary Metric.

A number of key metrics are now analyzed for an Ensemble model, where its constituent models have been selected based on best performing Log Loss out of a cohort of trained AI models. This particular Ensemble model had a voting strategy of Majority-Max confidence. Then a breakdown of the model performance associated with these metrics by class is also considered.

The same shared validation set of 252 images and blind test set of 527 images from the previous section is used.

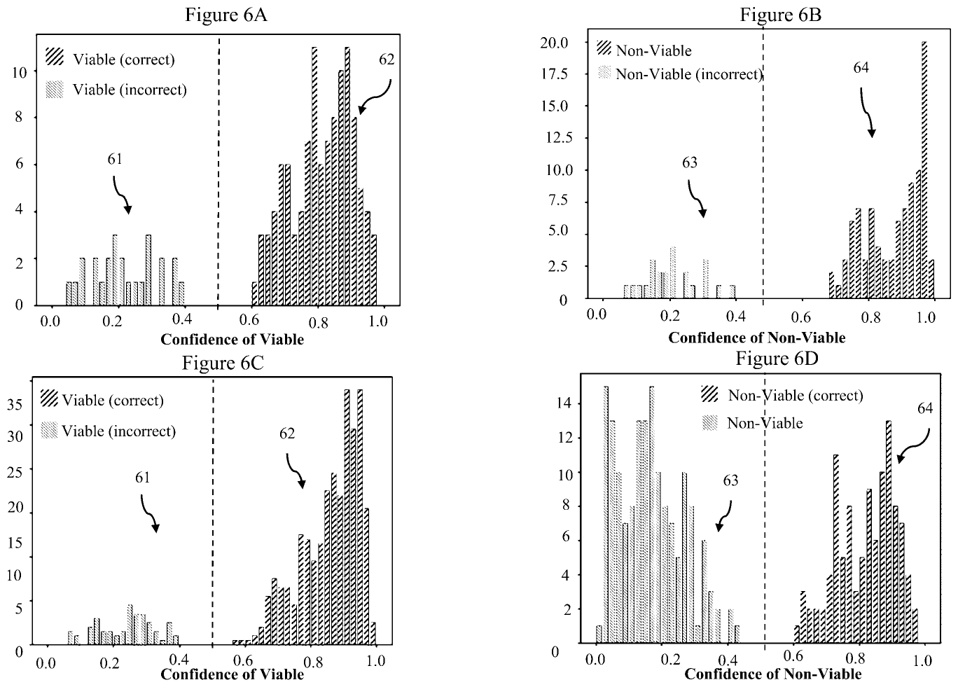

The histogram associated with the scores assigned by the model to the viable embryos on the shared validation set can be seen in FIG. 6A where correctly classified embryos are colored as bars with thick forward diagonal lines (True Positives) 62, and incorrectly classified embryos are colored as bars with thin rearward diagonal lines (False Negatives) 61. The equivalent histogram for non-viable embryos is shown in FIG. 6B where correctly classified embryos are colored as bars with thick forward diagonal lines (True Negatives) 64, and incorrectly classified embryos are colored as bars with thin rearward diagonal lines (False Positives) 63. The model distribution is extremely well-separated, and a high value of TPR and TNR. This is due both to the constituent models being selected based on the metric Log Loss, which takes into account distribution information, and tends to prioritize models that exhibit a high degree of separation, and also the optimal voting strategy for this model being max-confidence, which also has a tendency to enhance the bi-modal aspect of the distribution.

The separation between the correctly and incorrectly identified embryos on the blind test set persists, and the distribution associated with the less-noisy viable embryos in FIG. 6C is consistent with the validation set in FIG. 6B. Again, in this scenario the value of Class 0 accuracy, or TNR/Specificity drops, as exhibited in FIG. 6D. The metrics associated with both the validation and blind test sets are considered in the next section on Class Breakdown.

Class Breakdown

Metrics associated with the breakdown of results for the two classes, viable and non-viable examples, are shown in Table 5, for all clinics represented in the combined validation set. While the Accuracy metrics and Log Loss both outperform the values in the previous section on Class breakdown (Tables 2 and 3), Table 6 shows a larger drop in the Accuracy for ‘Class 0,’ or non-viable embryos due to label noise when applied to the blind test set, and outperforms the model of the section on metrics for Ensemble model with constituent AI models chosen based on Balanced Accuracy in terms of Accuracy for ‘Class 1’ or viable embryos. Note, however, that the Log Loss deteriorates due to the reduction in the non-viable accuracy but remains at a fair value.

As in the case above, the class-specific Log Losses are also compared and combined, and it is found that the selection of models based on these metrics are consistent with Log Loss.

TABLE 5
The class breakdown of the Ensemble model using Log Loss as the primary metric is shown on a
shared validation set, including the mean, balanced and combined class metrics.

| | | | Class 0 | Class 1 | | |
|---|---|---|---|---|---|---|
| | Mean class | Balanced | Accuracy | Accuracy | | |
| Clinic | accuracy | Accuracy | (TPR) | (TNR) | F1-Score | Log Loss |
| Combined | 80.95238 | 80.91168 | 80.34188 | 81.48148 | 0.80911681 | 0.45646541 |

TABLE 6
The class breakdown of the Ensemble model Log Loss as the primary metric is shown on a shared
blind test set including the mean, balanced and combined class metrics.

| | | | Class 0 | Class 1 | | |
|---|---|---|---|---|---|---|
| | Mean class | Balanced | Accuracy | Accuracy | | |
| Clinic | accuracy | Accuracy | (TPR) | (TNR) | F1-Score | Log Loss |
| Combined | 73.6242884 | 69.119554 | 53.3877754 | 84.8513327 | 0.69119554 | 0.5726757 |

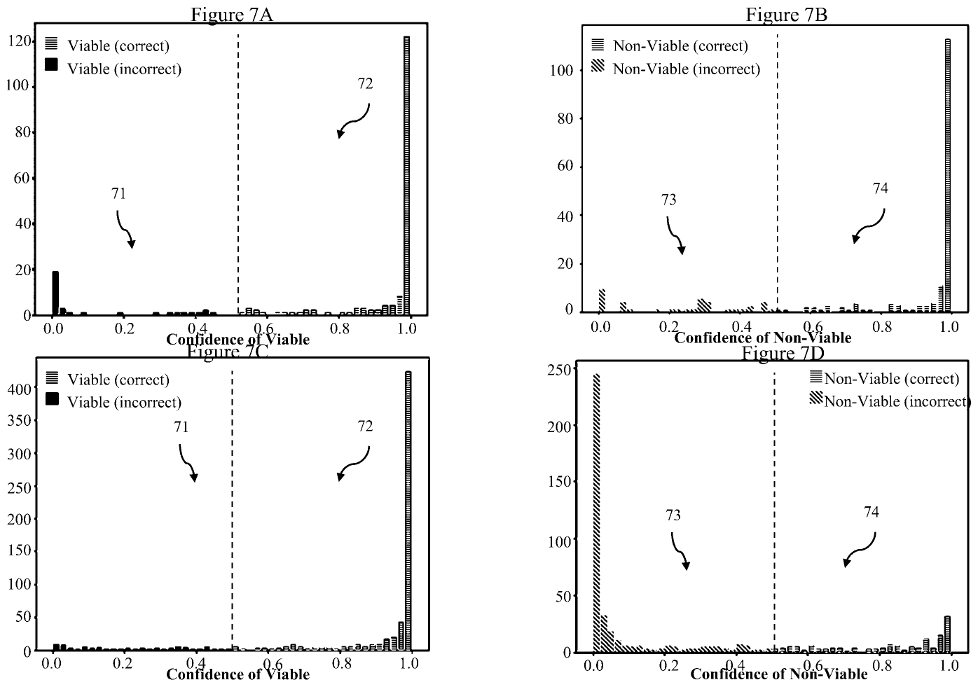

As a further example, FIGS. 7A to 7D show histograms obtained using Per-Class Ratio of Tangent Score vs Log Loss as the primary metric. FIGS. 7A and 7C represent the histograms of the ratios, from 0.0 to 1.0, with a binary threshold of 0.5, for viable embryos (indicated by vertical dashed line). Correctly classified embryos are shown as bars with thick horizontal lines (True Positives) 72, and incorrectly classified embryos are shown as black columns (False Negatives) 71. FIGS. 7B and 7D show the equivalent histograms for the non-viable embryos, where correctly classified embryos are shown as bars with horizontal lines (True Negatives) 74, and incorrectly classified embryos are shown as bars with thick rearward diagonal lines (False Positives) 73. Again these show the model separations are well-separated further illustrating the benefits of a confidence based metric. It can also been seen in these histograms that False Negatives are minimized by using the Log Loss metric in the class (Viable embryos) which is considered to be less noisy (less incorrectly-labelled examples), thereby ensuring that the model does not allow many examples of False Negatives. False Negatives (misclassifying a viable embryo as non-viable) in the case of embryo viability are considered to be a higher-risk misclassification compared to False Positives (misclassifying a non-viable embryo as viable). In the case of False Positives, the Tangent Score metric tolerates a certain quantity of noise/misclassified examples if they are offset by a similar number of correctly-classified examples at the same level of confidence. Therefore, the class which is considered to be more noisy (more incorrectly-labelled examples, such as those that appear non-viable, but actually are viable, and were misclassified due to patient medical conditions outside the embryo image) has a lessened effect of causing viable embryos to be misclassified because of the noise. The model training therefore obtains a superior result during validation and testing, because its training phase is more robust to noise.

As discussed previously, most AI training methods focus on total accuracy, or variations of total accuracy to judge the performance of a model. These may include the accuracy of the model on individual classes (of the categories of classification), i.e., ‘class accuracy,’ and accuracy variants, such as weighting the accuracy by the total number of images in each category or class, i.e., ‘balanced accuracy.’ However a problem with these accuracy focused metrics is that the translatability or generalizability of the AI model are not directly measured by these quantities.

In contrast the embodiments discussed herein can be used to create well-performing AI model that are guided by both accuracy (for final reporting) and level of confidence (or distribution of the level of confidence/score) that the AI model can classify certain images/data correctly. In particular, the methods incorporate one or more metrics that measures this level of confidence correctly as an intermediate step in selecting the best AI model among many potential models, prior to reporting.

In particular the method proposes calculating multiple metrics for a range of models on the same validation set and using these results to select top performing and/or diverse model configurations in an ensemble model. After selection, the model is then applied to a blind or double-blind test set and the performance of the model on the blind sets with respect to multiple metrics assessed. We comment that a well-generalizing model would be expected to have a high Accuracy metric on blind test sets, even if it was not selected using the metric of Accuracy. Selecting a model based on another metric may indeed lead to improved performance in not only that metric, but also other metrics that are more commonly reported and understandable to people outside the field of AI, such as Accuracy.

It is noted that the final report of accuracy on a validation or test set may in fact be lower for a well-performing model than a counterpart model that has been over-trained on a distribution of data from which the validation or test set have been sourced. However selecting models for commercial use, or for combining together using an Ensemble model approach, where confidence in accurate classifications is considered, reduces uncertainty and creates more robust models compared with selecting models based on a binary accuracy metric where the image confidence/score sits on either side of an arbitrary threshold of 50% (or more generally, in the case where a correct score for a class is just above the confidence scores for other classes). For example, achieving 100% accuracy in correctly classifying 1000 (blind test) images with an AI score/confidence of 55% (given a threshold of 50% as a correct classification) is likely to be of lesser value than achieving 100% accuracy in correctly classifying 1000 images with an AI score/confidence of 99.9%.

As outline above to select the AI Model constituents that will form the final model, the performance of each model at each training epoch is assessed on their shared validation set, using a primary metric, and we then select two or more (or all) of the trained AI models for inclusion in the ensemble model based on the stored best primary metrics. For example in the above embodiment which the AI model is a Day 5 embryo viability binary classification based model, the primary metric is Log Loss. Although other metrics are considered apart from the primary metric, and information regarding the training process which has taken place, plots describing the loss per epoch, and the distribution of the scores at each epoch, the primary metric is used as the first metric for sorting the performance of models for selection, or as candidates for inclusion in an Ensemble model.

Various embodiments for generating AI models based on confidence metrics have been described. These methods train a plurality of AI models on a common validation dataset over many epochs. A confidence metric as the best epoch (over all the epochs) is saved to allow comparison of the different AI models. A final AI model can then be selected using these AI models, for example using ensemble, distillation or other selection methods. In the case of an ensemble model, a confidence based voting strategy may be used. The experimental results show that confidence metrics such as Log Loss or its associated metrics (e.g., Combined Class Log Loss, Combined Data-Source Log Loss, Combined Class and Data-Source Log Loss, tangent score, bounded tangent score, per-class ratio of tangent score vs Log Loss, and Sigmoid score), which consider both accuracy in correctly classifying data and confidence in the AI in correctly classifying data (i.e., the AI score for correct classifications are high, exhibiting confidence in the correct classification), will result in more accurate and generalizable models that can be applied in wide variety of contexts including healthcare

Models incorporating confidence metrics are more robust and more reliable, because a greater confidence in correct classifications implies that the AI model has identified features or correlations more strongly across the broader dataset for each class and data source, making it less susceptible to variations or outliers in new unseen data.

Models that are selected using confidence metrics, whilst they may exhibit a reduction in accuracy in the validation dataset, have been demonstrated to have a higher final accuracy overall when applied to a blind (unseen) test set.

The results presented here demonstrate that models selected using this methodology therefore exhibit superior generalizability, less prone to overfitting, and therefore represent superior models as a result of this selection procedure, compared with other models trained on the same dataset.

Embodiments of the method can be used in healthcare applications (e.g., on healthcare data), and in particular healthcare datasets comprising images captured from a wide range of devices such as microscopes, cameras, X-ray, MM, etc. Models trained using embodiments discussed herein may be deployed to assist in making various healthcare decisions, such as fertility and IVF decisions and disease diagnosis. However it will be understood that the methods can also be used outside of the healthcare environment.

Those of skill in the art would understand that information and signals may be represented using any of a variety of technologies and techniques. For example, data, instructions, commands, information, signals, bits, symbols, and chips may be referenced throughout the above description may be represented by voltages, currents, electromagnetic waves, magnetic fields or particles, optical fields or particles, or any combination thereof.

Those of skill in the art would further appreciate that the various illustrative logical blocks, modules, circuits, and algorithm steps described in connection with the embodiments disclosed herein may be implemented as electronic hardware, computer software or instructions, middleware, platforms, or combinations of both. To clearly illustrate this interchangeability of hardware and software, various illustrative components, blocks, modules, circuits, and steps have been described above generally in terms of their functionality. Whether such functionality is implemented as hardware or software depends upon the particular application and design constraints imposed on the overall system. Skilled artisans may implement the described functionality in varying ways for each particular application, but such implementation decisions should not be interpreted as causing a departure from the scope of the present invention.

The steps of a method or algorithm described in connection with the embodiments disclosed herein may be embodied directly in hardware, in a software module executed by a processor, or in a combination of the two, including cloud based systems. For a hardware implementation, processing may be implemented within one or more application specific integrated circuits (ASICs), digital signal processors (DSPs), digital signal processing devices (DSPDs), programmable logic devices (PLDs), field programmable gate arrays (FPGAs), processors, controllers, micro-controllers, microprocessors, or other electronic units designed to perform the functions described herein, or a combination thereof. Various middleware and computing platforms may be used.

In some embodiments the processor module comprises one or more Central Processing Units (CPUs) or Graphical processing units (GPU) configured to perform some of the steps of the methods. Similarly a computing apparatus may comprise one or more CPUs and/or GPUs. A CPU may comprise an Input/Output Interface, an Arithmetic and Logic Unit (ALU) and a Control Unit and Program Counter element which is in communication with input and output devices through the Input/Output Interface. The Input/Output Interface may comprise a network interface and/or communications module for communicating with an equivalent communications module in another device using a predefined communications protocol (e.g., Bluetooth, Zigbee, IEEE 802.15, IEEE 802.11, TCP/IP, UDP, etc.). The computing apparatus may comprise a single CPU (core) or multiple CPU's (multiple core), or multiple processors. The computing apparatus is typically a cloud based computing apparatus using GPU clusters, but may be a parallel processor, a vector processor, or be a distributed computing device. Memory is operatively coupled to the processor(s) and may comprise RAM and ROM components, and may be provided within or external to the device or processor module. The memory may be used to store an operating system and additional software modules or instructions. The processor(s) may be configured to load and executed the software modules or instructions stored in the memory.

Software modules, also known as computer programs, computer codes, or instructions, may contain a number a number of source code or object code segments or instructions, and may reside in any computer readable medium such as a RAM memory, flash memory, ROM memory, EPROM memory, registers, hard disk, a removable disk, a CD-ROM, a DVD-ROM, a Blu-ray disc, or any other form of computer readable medium. In some aspects the computer-readable media may comprise non-transitory computer-readable media (e.g., tangible media). In addition, for other aspects computer-readable media may comprise transitory computer-readable media (e.g., a signal). Combinations of the above should also be included within the scope of computer-readable media. In another aspect, the computer readable medium may be integral to the processor. The processor and the computer readable medium may reside in an ASIC or related device. The software codes may be stored in a memory unit and the processor may be configured to execute them. The memory unit may be implemented within the processor or external to the processor, in which case it can be communicatively coupled to the processor via various means as is known in the art.

Further, it should be appreciated that modules and/or other appropriate means for performing the methods and techniques described herein can be downloaded and/or otherwise obtained by computing device. For example, such a device can be coupled to a server to facilitate the transfer of means for performing the methods described herein. Alternatively, various methods described herein can be provided via storage means (e.g., RAM, ROM, a physical storage medium such as a compact disc (CD) or floppy disk, etc.), such that a computing device can obtain the various methods upon coupling or providing the storage means to the device. Moreover, any other suitable technique for providing the methods and techniques described herein to a device can be utilized.

The methods disclosed herein comprise one or more steps or actions for achieving the described method. The method steps and/or actions may be interchanged with one another without departing from the scope of the claims. In other words, unless a specific order of steps or actions is specified, the order and/or use of specific steps and/or actions may be modified without departing from the scope of the claims.

Throughout the specification and the claims that follow, unless the context requires otherwise, the words “comprise” and “include” and variations such as “comprising” and “including” will be understood to imply the inclusion of a stated integer or group of integers, but not the exclusion of any other integer or group of integers.

The reference to any prior art in this specification is not, and should not be taken as, an acknowledgement of any form of suggestion that such prior art forms part of the common general knowledge.

It will be appreciated by those skilled in the art that the disclosure is not restricted in its use to the particular application or applications described. Neither is the present disclosure restricted in its preferred embodiment with regard to the particular elements and/or features described or depicted herein. It will be appreciated that the disclosure is not limited to the embodiment or embodiments disclosed, but is capable of numerous rearrangements, modifications and substitutions without departing from the scope as set forth and defined by the following claims.

In general, in the following claims, the terms used should not be construed to limit the claims to the specific embodiments disclosed in the specification and the claims, but should be construed to include all possible embodiments along with the full scope of equivalents to which such claims are entitled.

1. A computational method for generating an Artificial Intelligence (AI) model, the method comprising:
training a plurality of Artificial Intelligence (AI) models using a common validation dataset over a plurality of epochs, wherein during training of each model, at least one confidence metric is calculated at one or more epochs, and, for each model, the best confidence metric value over the plurality of epochs, and the associated epoch number at the best confidence metric is stored;
generating an AI model comprising:
selecting at least one of the plurality of trained AI models based on the stored best confidence metric;
calculating a confidence metric for the selected at least one trained AI model applied to a blind test set; and
deploying the AI model if the best confidence metric exceeds an acceptance threshold.

2. The method as claimed in claim 1, wherein the at least one confidence metric is calculated at each epoch.

3. The method as claimed in claim 1 wherein generating an AI model comprises generating an ensemble AI model using at least two of the plurality of trained AI models based on the stored best confidence metrics, and the ensemble model uses a confidence based voting strategy.

4. The method as claimed in claim 3 wherein generating an ensemble AI model comprises:
selecting at least two of the plurality of trained AI models based on the stored best confidence metric;
generating a plurality of distinct candidate ensemble models wherein each candidate ensemble model combines the results of the selected at least two of the plurality of trained AI models according to a confidence based voting strategy;
calculating the confidence metric for each candidate ensemble model applied to a common ensemble validation dataset;
selecting a candidate ensemble model from the plurality of distinct candidate ensemble models and calculating a confidence metric for the selected candidate ensemble model applied to a blind test set.

5. The method as claimed in claim 4, wherein the common ensemble validation dataset is the common validation dataset.

6. The method as claimed in claim 4, wherein the common ensemble validation dataset is an intermediate test set not used in training the plurality of Artificial Intelligence (AI) models.

7. The method as claimed in claim 4, wherein the confidence based voting strategy is selected from the group consisting of maximum confidence, mean confidence, majority-mean confidence, majority-max confidence, median confidence, or weighted mean confidence.

8. The method as claimed in claim 1, wherein generating an AI model comprises generating a student AI model using a distillation method to train the student model using at least two of the plurality of trained AI models using at least one confidence metric.

9. The method as claimed in claim 1, wherein selecting at least one of the plurality of trained AI models based on the stored best confidence metric comprises: selecting at least two of the plurality of trained AI models, comparing each of the at least two of the plurality of trained AI models using a confidence based metric, and selecting the best trained AI models based on the comparison.

10. The method as claimed in claim 1 wherein, at least one confidence metric comprises one or more of Log loss, combined class Log loss, combined data-source Log loss, combined class and data-source Log loss.

11. The method as claimed in claim 1, wherein a plurality of assessment metrics are calculated and are selected from the group consisting of accuracy, Mean class accuracy, sensitivity, specificity, a confusion matrix, Sensitivity-to-specificity ratio, precision, negative predictive value, balanced accuracy, Log loss, combined class Log loss, combined data-source Log loss, combined class and data-source Log loss, tangent score, bounded tangent score, per-class ratio of tangent score vs Log Loss, Sigmoid score, epoch number, mean of square error (MSE), root MSE, mean of average error, mean average precision (mAP), confidence score, Area-Under-the-Curve (AUC) threshold, Receiver Operating Characteristic (ROC) curve threshold, Precision-Recall curve

12. The method as claimed in claim 11, wherein the plurality of assessment metrics comprises a primary metric and at least one secondary metric, wherein the primary metric is a confidence metric, and the at least one secondary metric are used as tiebreaker metrics.

13. The method as claimed in claim 1, wherein the plurality of AI models comprise a plurality of distinct model configurations, wherein each model configuration comprises a model type, a model architecture, and one or more pre-processing methods.

14. The method as claimed in claim 13, wherein the one or more pre-processing methods comprises segmentation, and the plurality of AI models comprises at least one AI model applied to unsegmented images, and at least one AI model applied to segmented images.

15. The method as claimed in claim 13, wherein the one or more pre-processing methods comprises one or more computer vision pre-processing methods.

16. The method as claimed in claim 1 wherein the validation dataset is a healthcare dataset comprising a plurality of healthcare images.

17. A computational system comprising one or more processors, one or more memories, and a communications interface, wherein the one or more memories store instructions for configuring the one or more processors to generate an Artificial Intelligence (AI) model by:
training a plurality of Artificial Intelligence (AI) models using a common validation dataset over a plurality of epochs, wherein during training of each model, at least one confidence metric is calculated at one or more epochs, and, for each model, the best confidence metric value over the plurality of epochs, and the associated epoch number at the best confidence metric is stored;
generating an AI model comprising:
selecting at least one of the plurality of trained AI models based on the stored best confidence metric;
calculating a confidence metric for the selected at least one trained AI model applied to a blind test set; and
deploying the AI model if the best confidence metric exceeds an acceptance threshold.

18. A computational system comprising one or more processors, one or more memories, and a communications interface, wherein the one or more memories are configured to:
store a final trained AI model, wherein the trained AI model is generated by a computational system configured to:
train a plurality of Artificial Intelligence (AI) models using a common validation dataset over a plurality of epochs, wherein during training of each model, at least one confidence metric is calculated at one or more epochs, and, for each model, the best confidence metric value over the plurality of epochs, and the associated epoch number at the best confidence metric is stored;
generate a final trained AI model comprising:
selecting at least one of the plurality of trained AI models based on the stored best confidence metric;
calculating a confidence metric for the selected at least one trained AI model applied to a blind test set; and
store the final trained AI model if the best confidence metric exceeds an acceptance threshold; and
and the one or more processors are further configured to receive input data via the communications interface, process the input data using the stored AI model to generate a model result, and the communications interface is configured to send the model result to a user interface or data storage device.
